(19) United States
(12) Patent Application Publication
(10) Pub. No.: US 2002/0082561 A1
(43) Pub. Date: Jun. 27, 2002

(54) Needleless fluid transfer apparatus and related methods

(76) Inventors: Alan Fujii, Newport Beach, CA (US); John Kenneth Zeis, San Marcos, CA (US)

Correspondence Address: David L. Henty, MYERS, DAWES & ANDRAS LLP, Suite 1150, 19900 MacArthur Blvd., Irvine, CA, 92612, US

(21) Appl. No.: 09/749,067

(22) Filed: Dec. 27, 2000

(51) Int. Cl. B29C 45/00; A61M 5/00

(52) U.S. Cl. 604/187; 264/328.1

(57) ABSTRACT

A needleless fluid transfer apparatus includes a housing and a plunger. The housing includes a punch pointed enough to penetrate a tube with a stopper, yet substantially blunt such that it would not puncture human skin. The plunger is movable between a forward, dispensing position and a retracted, retaining position with respect to the housing. The plunger includes a piston with a seal that helps to retrieve fluid into a fluid passageway inside the housing. A projection extending distally from the seal minimizes dead volume by expelling fluid which would otherwise be disposed within a punch of the housing. The external structure of the plunger is designed for easy maneuverability by the user such that the apparatus may be operated with a single hand. A positive stop is provided such that exact volume of fluid may be retrieved and dispensed.

### BACKGROUND OF THE INVENTION

1. Field of the Invention

The present invention relates generally to safety syringes and the transfer of fluids from a tube. The invention further relates to methods of manufacture of fluid transfer devices.

2. Description of the Prior Art and Related Information

The transfer of fluids from one apparatus to another, such as from a tube to a testing apparatus, is necessary for a variety of applications in the medical and scientific fields. The prior art includes the standard syringe with a needle. With the spread of diseases and viruses, needles can often be dangerous as they are prone to prick the fingers of a user. Different means of shielding the needle of a syringe haven been developed, including protective shields, retractable needles and more. In addition to the danger of puncturing the skin, conventional syringes are problematic in that they do not provide for a fixed volume delivery. Should an exact volume of liquid be required, a user of a conventional syringe would have to visually gauge the barrel of the syringe to determine when to cease drawing in liquid.

The prior art also includes needleless devices designed for the transfer of fluids. Though these devices typically cannot puncture the skin, such needleless devices have the same drawback as conventional syringes in that they do not provide means for exact volume delivery, which may be significant for certain procedures. Representative of such needleless art are a number of devices that include an injection portion with a sharp tip and an attached bulb for suctioning in and dispensing liquid.

The prior art also includes pipettes. Since pipettes are unable to penetrate, for example, the stopper of a tube, the use of pipettes requires that the stopper first be manually removed. Removing stoppers, however, can increase the possibility of spillage and/or contamination of the fluid. In order to attempt a fixed volume delivery, dispensed drops must be counted or the pipette must be filled to a predetermined level, either of which requires gauging by the user and thus increases chances of error and variation.

Furthermore, conventional fluid transfer devices are typically composed of a plurality of components which increase costs and decrease manufacturability. The complexity of such devices also leads to inconvenience as a user is required to use both hands for operation. Retrieving liquid from a tube can be very difficult with conventional fluid transfer devices since a user must apply both hands to operate the device while somehow holding the tube at the same time.

Accordingly, a need presently exists for an improved fluid transfer device which overcomes some or all of the above-noted deficiencies.

### SUMMARY OF THE INVENTION

The present invention provides structures and methods which overcome the deficiencies in the prior art.

In one aspect, a fluid transfer apparatus adapted to be held and operated in one hand of a user is provided. The apparatus comprises a housing and a plunger. The housing comprises a fluid reservoir, a proximal end, a distal end, and a tapered punch disposed at the distal end having a blunt tip. The plunger is disposed at least in part within the housing. The plunger comprises a seal disposed within the housing. The plunger is movable with respect to the housing between a forward position occupying substantially all of the fluid reservoir and a retracted, fluid receiving position. The seal is in sliding frictional engagement with an inner wall of the housing. The seal comprises an annular flange that may be tapered.

The housing comprises a fluid reservoir defined by the punch and a tube disposed proximally adjacent to the punch. The inner wall of the housing comprises an inner surface of the tube such that the seal is in sliding frictional engagement with the inner surface of the tube. The fluid reservoir comprises a punch cavity defined by the punch and a tube cavity defined by the tube. The plunger comprises a piston adapted to be disposed at least in part within the punch cavity of the fluid reservoir when the plunger is in the forward position. The piston substantially displaces all fluid in the punch cavity when the plunger is in the forward position. The piston comprises a piston rod and a tapered projection. The piston rod is adapted to be substantially disposed within the tube when the plunger is in the forward position. The tapered projection is adapted to be substantially disposed within the punch when the plunger is in the forward position. The seal is disposed between the proximal portion and the tapered distal portion of the rod. The plunger further comprises a finger engagement member. The apparatus further comprises a stop mechanism that defines the retracted position.

In a preferred embodiment, the housing and the plunger comprise the only components of the apparatus.

In another aspect, a two-piece syringe assembly is provided. The assembly comprises a first unitary component and a second unitary component. The first unitary component comprises a punch with a blunt tip and a fluid reservoir for receiving liquid. The second unitary component is disposed at least in part within the first component and movable with respect to the first molded component. The second unitary component is movable with respect to the first unitary component between a forward position, whereby no liquid is retained, and a retracted position, whereby liquid is retained. The second component has a seal disposed within the retention cavity. The fluid reservoir defines a fixed volume. The second unitary component comprises a projection extending distally from the seal. The second unitary component comprises a flange and the first unitary component comprises a corresponding butting surface. The flange and corresponding butting surface form a stop that defines the retracted position. In a preferred embodiment, the seal is tapered. The second unitary component comprises an engagement mechanism for moving the second unitary component. The second unitary component preferably comprises the only other component of the assembly.

In another aspect, a fluid transfer apparatus comprises a housing and a plunger. The housing comprises a fluid retention portion with a tube and a punch. The punch has an inner surface. The plunger is movable with respect to the housing. The plunger comprises a projection with an outer surface that conforms substantially to the inner surface of the punch. Thus, the distal projection of the plunger conforms to a cavity defined by the punch so as to expel any fluid retained in the punch cavity.

A method for manufacturing and assembling a sample transfer apparatus is also provided. The method comprises: molding a first unitary component comprising a housing with a punch, the punch having a blunt tip on a distal end thereof, the housing defining a fluid reservoir; molding a second unitary component comprising a plunger with a seal adapted to frictionally slide along an inner wall of the housing, the plunger being disposed at least in part within the housing, the seal being disposed within the fluid reservoir; and configuring the plunger to be movable with respect to the seal.

Molding the first unitary component comprises forming the fluid reservoir to retain a fixed volume of liquid. The method further comprises: providing the plunger with a stop; adjusting the stop to adjust a volume capacity of the apparatus; tapering the seal; and providing a projection extending distally from the seal. Molding the second unitary component comprises molding the only other component of the apparatus.

In summary, a needleless fluid transfer apparatus, adapted to be held and operated by one hand of a user, includes a housing and a plunger. The housing includes a punch pointed enough to penetrate a tube with a stopper, yet substantially blunt such that it would not puncture human skin with normal operating force. The plunger is movable between a forward, dispensing position and a retracted, retaining position with respect to the housing. The plunger includes a piston with a seal that helps to retrieve fluid into a fluid passageway inside the housing. A projection extending distally from the seal minimizes dead volume by expelling fluid which would otherwise be disposed within a punch of the housing. The external structure of the plunger is designed and dimensioned such that the apparatus may be operated with a single hand. A positive stop is provided such that exact volume of fluid may be retrieved and dispensed.

The invention, now having been briefly summarized, may be better appreciated by the following detailed description.

### BRIEF DESCRIPTION OF THE DRAWINGS

FIG. 1 is a front perspective, exploded view of a fluid transfer apparatus according to the invention;

FIG. 2 is a front perspective view of the fluid transfer apparatus;

FIG. 3 is a rear perspective view of the fluid transfer apparatus;

FIG. 4 is a top plan view of the fluid transfer apparatus;

FIG. 5 is a rear plan view of the fluid transfer apparatus;

FIG. 6 is a cross-sectional side view of the fluid transfer apparatus in operation with the plunger in a forward position;

FIG. 7 is cross-sectional side view of the fluid transfer apparatus in operation with the plunger in a retracted position;

FIG. 8 is cross-sectional side view of the fluid transfer apparatus in operation with the plunger in a forward position;

FIG. 9 is a side view of a piston of the plunger;

FIG. 10 is a perspective view of an alternate embodiment of the fluid transfer apparatus;

FIG. 11 is a perspective view of a further alternate embodiment of the fluid transfer apparatus.

### DETAILED DESCRIPTION OF THE PREFERRED EMBODIMENTS

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

FIG. 1 is an exploded, frontal perspective view of a fluid transfer apparatus 10 according to the invention. The apparatus 10 may also be referred to as a safety sampler, a fluid transfer device, a fluid transfer assembly, or a safety syringe. The apparatus 10 comprises a first component, or housing, 12 and a second component, or plunger, 14. The first and second components 12, 14 are movable relative to each other and designed to fit together in a shape adapted for operation by a single hand of a user. The first and second components 12, 14 may comprise a molded plastic or otherwise formed material adapted for ease of manufacture. The housing 12 and the plunger 14 preferably comprise the only two molded components of the apparatus 10, thus resulting in a uniquely simple and advantageous two-piece assembly.

The housing 12 has a proximal end 20, a distal end 22, a front side 24 and a rear side 26. The housing 12 defines a fluid retention portion 30 for storing fluids retrieved into the apparatus 10. The fluid retention portion 30 comprises a punch 32 and an integral tube, or barrel, 34. The hollow punch 32 defines an inner punch cavity 31 and the tube 34 defines an inner tube cavity 35. The inner punch cavity 31 and the inner tube cavity 35 collectively form a fluid retention passageway, or fluid reservoir, 36, shown in FIG. 3, in which fluid retrieved by the apparatus 10 is disposed. An annular barrier 37, shown in FIG. 3, is disposed in the passageway 36 between inner punch cavity 31 and the inner tube cavity 35 to help create a vacuum when the plunger 14 is being moved.

In a preferred embodiment, the punch 32 is tapered, or conical, in that it narrows as it extends distally to a pointed, yet blunt tip 38. It should be understood that the tip 38 is referred to as blunt in that it will not puncture the skin of the user when operated with normal force while, at the same time, it is pointed in that it can penetrate, for instance, the rubber stopper of a tube, such as a vacuum tube. The inner punch cavity 31 is also tapered, or conical. The tip 38 comprises an aperture 39 through which fluid is retrieved into and dispensed from the apparatus 10. The tube 34 is preferably prismatic. As examples and not by way of limitations, the outer diameter of the tip may be in the range of ______ while the outer diameter of the base 33 of the punch 32 may be in the range of ______. For ease of operation by a single hand of the user, the other preferred dimensions of the apparatus 10 are ______.

Disposed proximally to the fluid retention chamber 30 is a majority portion 40 of the housing 12. The majority portion 40 comprises a hollow connector portion 42 that adjoins the retention chamber 30 and a base 44. In a preferred embodiment, as the housing 12 extends from the proximal end 20 to the distal end 22, each distally adjacent section or portion becomes smaller and narrower. The base 44 includes a shoulder 46 that provides an area for placing the user's index finger and middle finger when dispensing fluid. In a preferred embodiment, the shoulder 46 is sloped proximally as it extends from a front wall 48 to a rear wall 50 of the base 44, thereby resulting in a recess 50. Referring to the rear view of FIG. 3, a narrow slot 52 is defined in the rear wall 50 for guiding a tongue of the plunger 14. The rear wall 50 also includes an abutting surface, or stop, 54 that defines the distal end of the slot 52. A bottom opening 56 of the base 44 is continuous with a front opening 58 defined by an edge 57 in the front wall 48 of the base 44 and a front wall 43 of the connector tube 42. The rear slot 52, bottom opening 56 and front opening 58 are configured to correspond to structural features of the plunger 14 which extend outside the housing 12. The housing base 44 further comprises a bottom edge 59. In a preferred embodiment, the housing 12 is composed of a rigid, transparent and easily molded material, such as plastic. Though it is not necessary, a transparent housing 12 is preferable in that it allows the user to see the fluid retrieved into the apparatus 10.

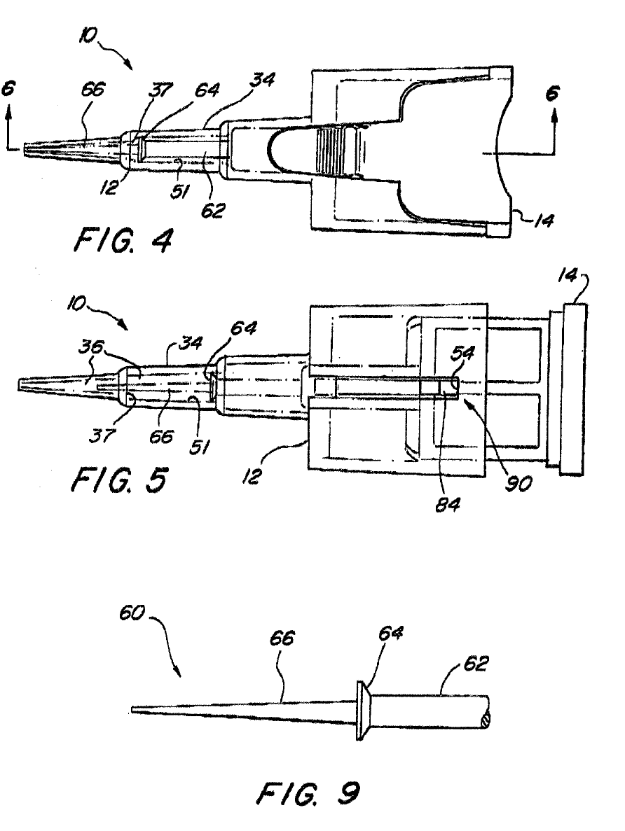

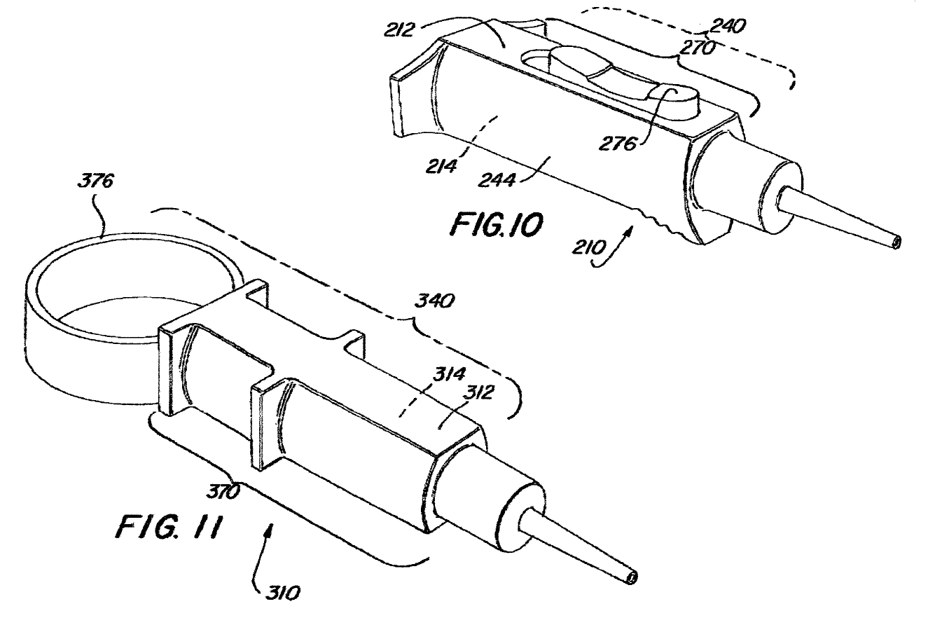

The plunger 14 comprises a piston 60. The piston 60 includes a piston rod 62, a piston head, or annular seal, 64. The annular seal 64, also shown in FIG. 9, has an inverted tapered profile so as to minimize surface contact with the inner surface 51 of the tube 34, shown in FIGS. 4 and 5. The seal 64 is integral with the remainder of the plunger 14 such that movement of the plunger 14 causes the seal 64 as well as all other components of the plunger 14 to move. The piston 60 further comprises a spear shaped projection 66 extending distally from the seal 64. The projection 66, also shown in FIG. 9, is conical such that it conforms to the conical inner cavity 31 of the punch 32. Alternatively stated, the projection 66 has an outer surface that conforms substantially to an inner surface of the punch 32.

Disposed proximally to the piston 60 is a connector portion 68. The connector portion 68 is coupled to a wide plunger base 70. The plunger base 70 includes a raised front portion 72 and a rear portion 74. The shape of the raised front portion 72 corresponds to the front opening 58 of the housing base 44. The front portion 72 further includes a grooved bump 76 for enabling a user to easily move the plunger 14 with his or her thumb. A flange 78 disposed at a proximal end 80 of the plunger 14 protrudes beyond the bottom edge 59 of the housing 12. The plunger 14 further comprises a concave bottom 82 conforming to the shape of a thumb. In FIGS. 1 and 3, a tongue 84 is disposed on a rear side of the plunger base 70 and configured to slide along the slot 52 of the housing 12.

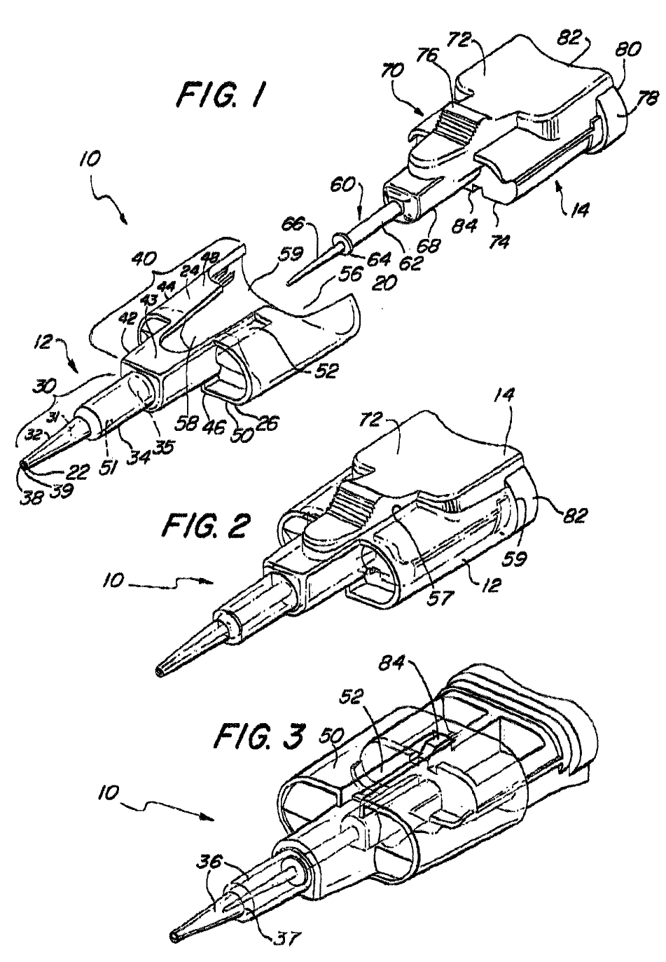

With respect to the housing 12, the plunger 14 is movable between a fully extended forward position as shown in FIG. 2 and fully retracted position shown in FIG. 3. Except for the raised front portion 72, the flanged bottom 82 and the tongue 84, the outer structure of the plunger 14 is configured to fit and slide within the inner structure of the housing 12. In the forward position, the raised front portion 72 of the plunger base 70 engages the front edge 57 of the housing 12 while the bottom flange 78 engages the bottom edge 59 of the housing, thereby limiting the plunger 14 from moving forward any further.

In FIG. 4, the apparatus 10 is shown with the plunger 14 disposed at a fully forward position with respect to the housing 12. The seal 64 is disposed at a distal end of the tube 34 adjacent to the annular barrier 37. The piston rod 62 is substantially disposed in the tube 34 while the projection 66 is disposed in the punch cavity 31. The plunger connector portion 68 is disposed within the housing connector portion 42, and the plunger base 70 is substantially disposed within the housing base 44. In the forward position, the apparatus 10 is used by inserting the punch 32 through the stopper of a fluid collection device, such as a vacuum tube, containing fluids.

The plunger 14 may be moved to a retracted position with respect to the housing 12 as shown in FIG. 5. As the plunger 14 is retracted, the tapered seal 64 being in frictional contact with an inner surface of the tube 34 creates an effective vacuum, thus drawing in liquid without creating any air pockets. The annular barrier 37 also helps maintain the vacuum. The passageway 36 is now substantially open for retaining fluids except for the projection 66 disposed therein. The plunger 14 is retracted until the tongue 84 contacts the abutting surface 54. Thus, the tongue 84 and abutting surface 54 collectively form a stop mechanism 90. In the retracted position, the seal 64 is disposed at a proximal end of the tube 34 while the projection 66 is at least partly disposed in the tube 34. Consequently, the plunger connector portion 68 is disposed within the housing base 44 and the plunger base 70 is at least partially disposed without the housing 12.

It will be appreciated that by altering the length of the slot 52, for instance, by adjusting the abutting surface 54, the fixed volume capacity may be altered. Thus, the simplified structure of the apparatus 10 allows for greater manufacturability in that the volume capacity of the apparatus 10 may be fixed at different levels by simply manufacturing the apparatus 10 with different slots 52.

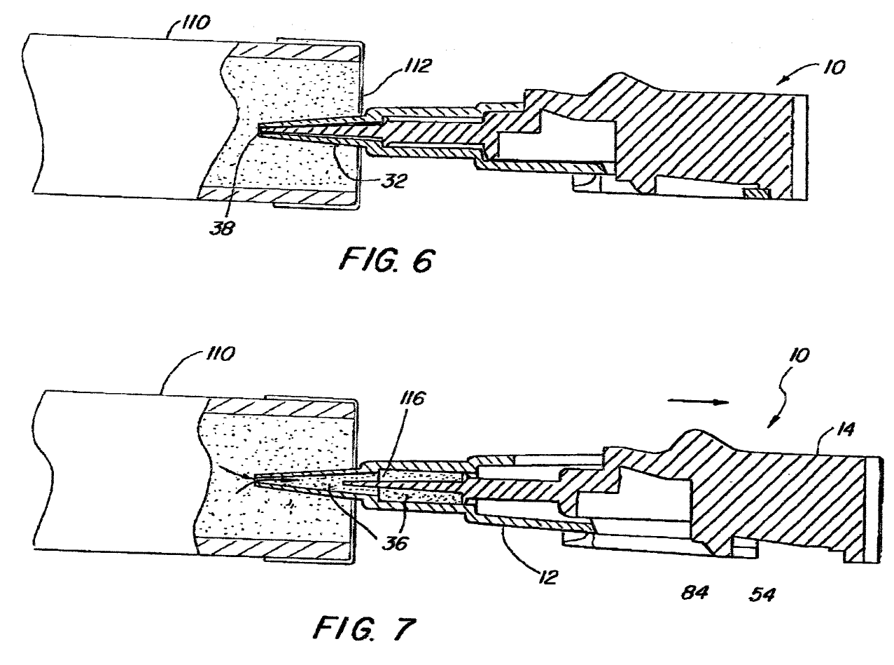

FIGS. 6-8 illustrate the apparatus 10 in operation. In the forward position shown in FIG. 6, the apparatus 10 may be coupled to a tube 110, such as a Vacutainer, containing fluid to be retrieved. The punch 32, having a pointed tip 38 but not enough so as to puncture human skin, is inserted through the stopper 112. In FIG. 7, the plunger 14 is moved to the retracted position. The seal 64 causes the fluid 116 to be drawn into the passageway 36. Exact volume retrieval may be accomplished by retracting the plunger 14 until it comes to a positive stop when its tongue 84 contacts the stopping surface 54 of the housing 12. The apparatus 10 may then be uncoupled from the tube 110. Unlike prior art syringes or pipettes, no measure of gauging is required by the user as the exact volume of fluid is accomplished when the plunger 14 is stopped.

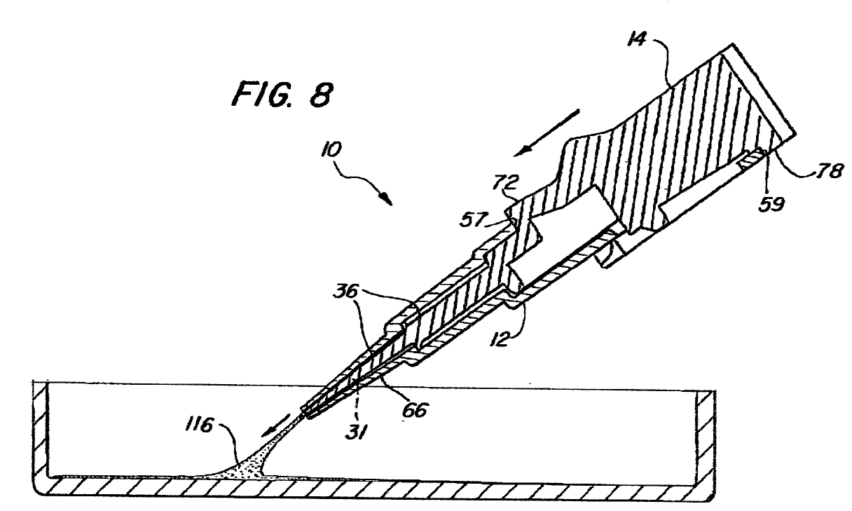

In FIG. 8, by moving the plunger 14 forward, such as by pushing the recessed bottom 82 of the plunger 14 with a thumb, both the seal 64 and the projection 66 dispense the retrieved fluid 116 out of the passageway 36. The forward movement of the plunger 14 is stopped by the raised front portion 72 and base flange 78 of the plunger 14 engaging the respective edges 57, 59 of the housing 12. Furthermore, the exterior structure of the plunger 14 engaging the interior structure of the housing 12 also serves to stop the forward movement of the plunger 14. It will be appreciated that the apparatus 10 minimizes “dead volume” in that the amount of fluid 116 dispensed is substantially equal to the amount 116 retrieved. This is accomplished by the projection 66 which expels fluid out of the passageway 36 that would otherwise remain in the punch cavity 31.

FIG. 9 is a close-up view of the piston 60. The piston head 64 has an inverted, tapered shape to minimize surface contact and, thereby, form a more effective seal. The distal projection 66 is conical while the piston rod 62 is cylindrical.

In FIGS. 10 and 11, alternate embodiments are shown wherein the majority portions 270, 370 of the housing 212, 312, respectively, and majority portions 240, 340 of the plungers 214, 314, respectively, are modified. The structure of the portions of housing 212, 312 distal to the majority portions 270, 370 are substantially similar to the first preferred embodiment illustrated in FIGS. 1-9. Similarly, the structure of the portions of the plunger 214, 314 distal to the majority portions 240, 340, are also substantially similar to the structure of the embodiment in FIGS. 1-9.

In FIG. 10, an alternate embodiment 210 is shown wherein the plunger 214 includes a button 276 for easy maneuvering by the user. For example, a user may grip the cylindrical housing base 244 in the palm of one hand while engaging the button 276 with the thumb of the same hand. In this embodiment, the housing base 244 is substantially cylindrical so that the user may easily grip the apparatus 10a with the palm of the hand while engaging the button 276 with the thumb of the same hand.

In FIG. 11, a further alternate embodiment 310 is provided wherein a loop 376 is coupled to the plunger 314 to enable the user to pull the plunger 314 back with the thumb.

A method for manufacturing and assembling a fluid transfer device is also provided. The method comprises: providing a housing with a punch; forming a blunt tip on a distal end of the punch; forming a retention cavity within the housing; providing a plunger with a seal adapted to frictionally slide along an inner wall of the housing; disposing the plunger at least in part within the housing; disposing the seal within the retention cavity; and configuring the plunger to be movable with respect to the seal.

Forming a retention cavity within the housing may comprise forming the retention cavity to retain a fixed volume of liquid. The method further comprises providing the plunger with a stop. The method further comprises adjusting the stop to adjust a volume capacity of the apparatus; tapering the seal; and providing a projection extending distally from the seal. Providing a housing with a punch comprises molding the housing. Providing a plunger with a seal comprises molding the plunger with the seal.

It will be appreciated that the apparatus according to the invention enables a user to retrieve and dispense fluid with merely one hand, thus freeing the other hand, for example, to hold a tube from which fluid is being withdrawn and/or a testing apparatus to which fluid is being dispensed.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what incorporates the essential idea of the invention.

What is claimed is:

1. A fluid transfer apparatus adapted to be held and operated in one hand of a user, comprising:
a housing comprising a fluid reservoir, a proximal end, a distal end, and a tapered punch disposed at the distal end having a blunt tip; and
a plunger disposed at least in part within the housing, the plunger comprising a seal disposed within the housing,
wherein the plunger is movable with respect to the housing between a forward position occupying substantially all of the fluid reservoir and a retracted, fluid receiving position.

2. The apparatus of claim 1, wherein the seal is in sliding frictional engagement with an inner wall of the housing.

3. The apparatus of claim 2, wherein seal comprises an annular flange.

4. The apparatus of claim 3, wherein the seal is tapered.

5. The apparatus of claim 1, wherein the housing comprises a fluid reservoir, the fluid reservoir being defined by the punch and a tube disposed proximally adjacent to the punch.

6. The apparatus of claim 5, wherein the inner wall of the housing comprises an inner surface of the tube such that the seal is in sliding frictional engagement with the inner surface of the tube.

7. The apparatus of claim 5, wherein:
the fluid reservoir comprises a punch cavity defined by the punch and a tube cavity defined by the tube; and
the plunger comprises a piston adapted to be disposed at least in part within the punch cavity of the fluid reservoir when the plunger is in the forward position.

8. The apparatus of claim 7 wherein the piston substantially displaces all fluid in the punch cavity when the plunger is in the forward position.

9. The apparatus of claim 7, wherein the piston comprises a piston rod and a tapered projection, the piston rod adapted to be substantially disposed within the tube when the plunger is in the forward position, the tapered projection adapted to be substantially disposed within the punch when the plunger is in the forward position.

10. The apparatus of claim 9, wherein the seal is disposed between the proximal portion and the tapered distal portion of the rod.

11. The apparatus of claim 1, wherein the plunger further comprises a finger engagement member.

12. The apparatus of claim 1, further comprising a stop mechanism that defines the retracted position.

13. The apparatus of claim 1, wherein the housing and the plunger comprise the only components of the apparatus.

14. A two-piece syringe assembly comprising:
a first unitary component comprising a punch with a blunt tip and a fluid reservoir for receiving liquid; and
a second unitary component disposed at least in part within the first component and movable with respect to the first molded component, the second component having a seal disposed within the retention cavity.

15. The assembly of claim 14, wherein the fluid reservoir defines a fixed volume.

16. The assembly of claim 15, wherein the second unitary component comprises a projection extending distally from the seal.

17. The assembly of claim 15, wherein the second unitary component is movable with respect to the first unitary component between a forward position, whereby no liquid is retained, and a retracted position, whereby liquid is retained.

18. The assembly of claim 17, wherein the second unitary component comprises a flange and the first unitary component comprises a corresponding butting surface, the stop and corresponding butting surface forming a stop mechanism that defines the retracted position.

19. The assembly of claim 14, wherein the seal is tapered.

20. The assembly of claim 14, wherein the second unitary component comprises an engagement mechanism for moving the second unitary component.

21. The assembly of claim 14, wherein the second unitary component comprises the only other component of the assembly.

22. A fluid transfer apparatus comprising:
a housing comprising a fluid retention portion, the fluid retention portion comprising a tube and a punch, the punch having an inner surface; and
a plunger movable with respect to the housing, the plunger comprising a projection with an outer surface that conforms substantially to the inner surface of the punch.

23. A method for manufacturing and assembling a sample transfer apparatus, the method comprising:
molding a first unitary component comprising a housing with a punch, the punch having a blunt tip on a distal end thereof, the housing defining a fluid reservoir;
molding a second unitary component comprising a plunger with a seal adapted to frictionally slide along an inner wall of the housing, the plunger being disposed at least in part within the housing, the seal being disposed within the fluid reservoir; and
configuring the plunger to be movable with respect to the seal.

24. The method of claim 23 wherein molding the first unitary component comprises forming the fluid reservoir to retain a fixed volume of liquid.

25. The method of claim 23 further comprising providing the plunger with a stop.

26. The method of claim 25 further comprising adjusting the stop to adjust a volume capacity of the apparatus.

27. The method of claim 23 further comprising tapering the seal.

28. The method of claim 23 further comprising providing a projection extending distally from the seal.

29. The method of claim 23 wherein molding the second unitary component comprises molding the only other component of the apparatus.
